(19) United States
(12) Patent Application Publication
(10) Pub. No.: US 2019/0204325 A1
(43) Pub. Date: Jul. 4, 2019

(54) Protein Chip, Kit and Preparation Method thereof for Detecting Abnormal Decarboxy Prothrombin in Serum

(71) Applicants: BEIJING YOUAN HOSPITAL, CAPITAL MEDICAL UNIVERSITY, Beijing (CN); BEIJING INSTITUTE OF HEPATOLOGY, Beijing (CN); Beijing Houde Tiancheng Biotech Ltd., Beijing (CN)

(72) Inventors: Aiying ZHANG, Beijing (CN); Ronghua JIN, Beijing (CN); Ning LI, Beijing (CN); Shengqi WANG, Beijing (CN); Yang KE, Beijing (CN)

(73) Assignees: BEIJING YOUAN HOSPITAL, CAPITAL MEDICAL UNIVERSITY, Beijing (CN); BEIJING INSTITUTE OF HEPATOLOGY, Beijing (CN); Beijing Houde Tiancheng Biotech Ltd., Beijing (CN)

(21) Appl. No.: 16/314,672

(22) PCT Filed: Jun. 29, 2017
(86) PCT No.: PCT/CN2017/090784

§ 371 Date: Jan. 1, 2019

(30) Foreign Application Priority Data
Jul. 1, 2016 (CN) 201610515896.6

(51) Int. Cl. G01N 33/574; G01N 33/566; G01N 33/58

(52) U.S. Cl. CPC G01N 33/57438; G01N 33/582; G01N 33/566

(57) ABSTRACT

A protein chip, kit and preparation method thereof for detecting abnormal decarboxy prothrombin in serum, a substrate carrier of the protein chip is provided with a plurality of detection subareas; wherein each detection subarea is used for detecting a serum sample, and is internally provided with a detection-spot area and a control-spot area, and the detection-spot area has a detection spot formed by spraying a trace amount of a DCP-specific antibody, the control-spot area has a control spot formed by spraying a bovine serum albumin; all the detection spots within one of the detection-spot area have the same material concentration, to form each of the detection spots, a total volume of 3-5 nl of the DCP-specific antibody with a concentration of 3-5 mg/mL is used; each of the detection spots is formed by a non-contact spotter, performing 6-10 spot sprays and spraying 300-500 pL in each spray.

### TECHNICAL FIELD

The invention relates to a protein detection technology, in particular to a protein chip, a kit and a preparation method thereof for detecting abnormal decarboxy prothrombin in serum.

### BACKGROUND ART

Des-r-carboxy-prothrombin (DCP) is an abnormal prothrombin produced by hepatocellular carcinoma. Compared with normal prothrombin, the molecular structure of DCP is characterized by that one or more glutamate (Glu) residues in the Gla domain are not fully carboxylated to Gla, thus losing the coagulation function. Normal prothrombin is inside the liver cell microsomes, and the 10 Glu residues at position 6, 7, 14, 16, 19, 20, 25, 26, 29 and 32 in the structure of the Gla Domain are carboxylated to Gla for being an activated prothrombin, by mainly relying on Vitamin K gamma glutamine carboxylase and coenzyme and Vitamin K reductase; and incomplete carboxylation of Glu residues of any above sites or a plurality of sites can lead to the DCP, making prothrombin lose blood coagulation function.

Studies show that DCP in serum of primary hepatocellular carcinoma is significantly increased. L U Fenglin etc. published the “Diagnostic value of des-gamma-carboxy-prothrombin for primary hepatocellular carcinoma” in Chinese Journal of Clinical Oncology, Issue 07, 2009. It revealed that DCP value is positively correlated with tumor size. Therefore, accurate monitoring of DCP level in serum is of great significance for clinicians to judge the prognosis, select treatment options and observe the efficacy. Since a large number of samples need to be tested every day for hospitals in clinical testing, high-throughput chip detection can greatly improve the detection efficiency. In other words, a low-cost, fast, efficient, accurate and high-throughput detection method is the best choice, but there is no report on high-throughput detection of DCP at present. Existing detection methods in the detection of serum DCP methods include the electrochemical luminescence immunoassay technology, liquid phase affinity immunoassay, immunoprecipitation, western blot, ELISA, etc. It is known by those skilled in the art that clinical serum is very complex, not only containing other markers of other liver cancers, but serum DCP to be tested is affected by various nonspecific physical adsorption or nonspecific binding, such as hemagglutinin, thrombin and cellulose and its analogues are prone to be interfered.

Therefore, in order to ensure the accuracy, sensitivity and specificity of the detection results, there is a common feature among the detection methods using immune principles mentioned above, that is to say, the amount of serum for each detection reaction is as low as 50-100 ul and as high as 3-5 ml. Correspondingly, a large number of antibodies are required for each detection, such as microporous plate chemiluminiscence immunoassay method disclosed in CN101377505 A, and each hole needs to be added with a coating buffer of 150 ul that contains DCP antibodies, with a serum sample of 50 ul required; although the minimum detection limit disclosed in that document can reach 4.37 mAU/ml, real clinical serum was not adopted to verify the specificity and accuracy of the test in the experiments recorded in that document, and the test data were obtained from artificial samples formed by adding standard substances into a normal serum. Moreover, these methods have detection time as long as 3-4 hours; if high-throughput chips are made according to these methods, then the size of the detection spot or capture spot for each sample will be very large and the cost very high, which will lose the significance of making an integrated chip, namely the purpose of high throughput and low cost cannot be achieved, and it is not realistic to popularize it in the outpatient service. More explorations are needed for the realization of high-throughput DCP detection.

### SUMMARY OF THE INVENTION

The invention, directed at vacancy of in the field of detecting serum DCP by protein chip, provides a high-throughput protein chip, kit and its preparation method thereof suitable for the detection of serum DCP, maximally saving antibodies, serum dosage in premise of guaranteeing the detection accuracy, sensitivity and specificity, suitable for clinical use, with the advantages of timesaving, economy, accuracy and convenience.

The technical scheme of the invention is as follows:

In an aspect, the invention provides a protein chip for detecting abnormal decarboxy prothrombin in serum, which is characterized in that:

a plurality of detection subregions are provided on the substrate carrier of the protein chip, and each of the detection subregions is configured to detect a serum sample; each of the detected subregions is provided with a detection spot area and a control spot area, the detection spot area has a detection spot formed by spraying a trace of DCP specific antibody, and the control spot area has a control spot formed by spraying bovine serum albumin; substances on all the detection spots have the same concentration in the same detection spot area; and
the total amount of DCP specific antibody for forming each of the detection spots is 3-5 nl, and the concentration thereof is 3-5 mg/mL; each of the detection spots is formed by a non-contact point sampler in 6-10 times and spraying 300-500 pl each time, and the diameter of the detection spot is 0.5-1 mm.

Preferably, each of the detection spot areas includes 4-8 mutually separated detection spots arranged in a row, and the control spot area includes 4-8 mutually and independently separated control spots arranged in a row; the detection spots and the control spots are arranged in two parallel columns.

Preferably, the length, width and thickness of the chip are 76.4 mm, 25.2 mm and 1 mm, and 10 detection subregions are provided on the substrate carrier.

Preferably, a protrusion is provided between the detection subregions as a physical partition.

Preferably, the specific monoclonal antibody of the DCP is a murine anti-human DCP.

In another aspect, the invention provides a preparation method of a protein chip for detecting abnormal decarboxy prothrombin, which is characterized in that:

the total amount of DCP-specific antibody for forming each of the detection spots is 3 nl, and the concentration thereof is 4 mg/ml; a detection spot is formed by spraying the DCP-specific antibody in 6-10 times and 300-500 pl each time, and the diameter of the detection spot is 0.5-1 mm.

Preferably, the temperature of the DCP-specific antibody used is 4-8° C. during the spraying process.

Preferably, spraying point samples is performed by the non-contact point sampler.

The invention also provides a kit for detecting abnormal decarboxy prothrombin in serum based on the protein chip mentioned above, which is characterized by including any of the protein chips mentioned above, an HRP-labeled prothrombin polyclonal antibody, and HRP chemiluminescent substrate liquid; the HRP-labeled prothrombin polyclonal antibody is a rabbit antibody, and the DCP-specific antibody fixed on the detection spot is originated from a different species.

A method for detecting abnormal decarboxy prothrombin in serum is characterized by the use of any above-mentioned protein chips and includes the steps of:

diluting the serum samples to be tested and dropping onto the detection subregion of the protein chip, after incubation, washing the detection subregion with PBST to remove the nonspecific binding substances;
adding HRP-labeled prothrombin antibody diluted with PBS, after incubation, washing with PBST to remove nonspecific binding substances; and
adding HRP substrate luminescent solution, and scanning protein chip by chemiluminescence scanner to obtain DCP luminescence pixel values in serum samples to be measured after dilution respectively.

The incubation refers to incubation at 37° C. for 30 minutes.

The invention provides a chemiluminescent protein chip for detecting DCP. Based on the sandwich reaction principle of antibody-antigen-antibody and chemiluminescence principle, a detection spot is formed by spraying the specific antibody of DCP on the chip, so as to bind the DCP (abnormal decarbonylation site of prothrombin) in serum, and control spots are provided at the same time.

The inventor found, according to the conventional detection method of the immune principle, the capture antibody is coated on the substrate of the chip, with 10 ul and a concentration of 4 mg/ml, (the detection spots of 5-10 mm in diameter) for each detection spot, and standard solutions with a series of gradient concentrations were added to the serum (standard serum 0 mAU/ml, 5 mAU mAU/ml, 10 mAU/ml, 20 mAU/ml, 40 mAU/ml, 80 mAU/ml, 160 mAU/ml, 320 mAU/ml, and 640 mAU/ml) as samples, making a standard curve, then 50 ul standard serum was added to each subregion, and the subregion was washed with PBST to remove the nonspecific binding. HRP-labeled prothrombin antibody diluted with PBS was added, incubated, and washed with PBST to remove nonspecific binding substances. HRP substrate luminescent solution was added, and then the protein chip was scanned by chemiluminescence scanner to obtain the pixel value. The standard curve was drawn with the concentration of the standard serum as the horizontal axis and the pixel value as the vertical axis, and the regression equation of the standard curve was generated. However, the results showed that the correlation coefficient of the regression equation of the standard curve was low, and the R² value was between 0.6 and 0.7 after being repeated for many times, indicating that the detection accuracy of the chip was unstable and subject to more interference. Another batch of test standard serum with different concentrations was used to verify the standard curve. The results obtained were significantly different from the concentration of test standard serum itself, and moreover, the detection values of the test standard serum with the concentration lower than 80 mAU/ml were repeatedly lower than 30 mAU/ml, showing false negative.

By chance, the inventor discovered that the correlation coefficient of the regression equation of the standard curve could be significantly changed by adjusting the quantity of capture antibody applied on and formed the detection spot and the size of the detection spot. It was further found that the correlation coefficient of the regression equation of the standard curve was increased step by step when the quantity of capture antibody was reduced from 10 ul step by step and the non-contact point sample method was adopted. When the quantity of capture antibody was less than 10 nl, the correlation coefficient R² of the regression equation was increased to 0.65-0.75, and the result of the test by the test standard serum was more and more accurate. However, in the experiment where the quantity of capture antibody continues to decrease, the detection correlation coefficient R² has a tendency to decrease.

It was not ideal for the correlation coefficient R² of the standard curve regression equation between 0.65 and 0.75. After a variety of attempts, the inventor accidentally found that the capture antibody was sprayed in a plurality of times, for example, the capture antibody of 3-5 nl was sprayed to the spot location in 6-10 times. It was found that the correlation coefficient R² of the standard curve equation produced by the chip was increased to more than 0.95. Accurate results were obtained by the test standard serum and clinical serum, and the minimum detection level could be as low as 4 mAU/ml.

To sum up, the protein chip of the present invention is directed at targets being inactive prothrombin formed by incomplete carboxylation of 10 Glu residues at position 6, 7, 14, 16, 19, 20, 25, 26, 29 and/or 32 in the structure Gla Domain. Incomplete carboxylation of any one or more of the above Glu residues may form DCP including prothrombin with abnormal decarboxylation of 10 Glu residues at position 6, 7, 14, 16, 19, 20, 25, 26, and 29 and/or 32 in serum. In order to provide high-throughput protein chip for accurately qualitative testing des-r-carboxy-prothrombin (DCP) of in serum, the inventor adjusted chip making process, including developing appropriate method of spotting the capture antibody on the chip, adjusting the capture antibody's concentration and volume, and adjusting the size of the detection spot to ensure the accuracy. It is speculated, the way of pointing the capture antibody by many times & mice volume each time enables the antibody adhere to the chip substrate uniformly and steadily, and reduced or avoids hollows existed in the detection spots; the size of the detection spots ensures a reasonable density of antibodies and provides the sufficient space for antibodies to maintain its spatial structure and activity; and at the same time it is not too sparse to result in too low pixel value for detection of low concentration sample and form false negative result The amount of capture antibody on the detection spot, the pointing times of the capture antibody, and the size of the detection spot have a direct impact on the detection results.

As shown in FIG. 1, in one preferred example of the present invention, a detection subregion 2 includes four detection spot 3 that fixed DCP-specific antibodies and four control spot 4. The detection spot 3 and the control spot 4 are placed in two parallel columns.

In a further aspect, the invention also provides a preparation method of the chip.

In one more aspect, the invention also provides a chemiluminescence kit for detecting DCP, which includes the protein chip mentioned above and a conventional chemiluminescence reagent.

The use of the protein chip of the invention has three advantages:

I. Detection of DCP in serum.

II. A plurality of samples are allowed to be tested simultaneously, such as a plurality of repeated samples, or samples taken at different time points to obtain dynamic values, or different samples, in short, to achieve high throughput detection, and the detection cost is reduced on the whole and the detection efficiency is improved.

III. The amount of blood samples and antibodies required for the protein chip of the invention are greatly reduced. Each spot is sprayed with 300-500 pl at a time for 6-10 times, totally 3 nl of the capture antibody per spot. Four detection spots are provided in each detection subregions, which required 12 nl of the capture antibody, so as to guarantee the homogeneity of the capture antibody and effectively reduce the occurrence of hollow phenomenon. In addition, only 10 ul of the original serum or diluted serum is required, while in the detection methods of prior arts, not only require a large amount of capture antibody, but also need at least 50 ul of the original serum or diluted serum.

Finally, the invention also provides a method for detecting serum DCP by above kit.

The method of detecting serum DCP provided by the invention is to combine the DCP antibody fixed on the chip with the DCP in serum or plasma (abnormal decarboxylation site of the abnormal decarboxy prothrombin) on the protein chip using the characteristics of specific binding of antibody and antigen. Then HRP-labeled prothrombin polyclonal antibody is added, and the prothrombin polyclonal antibody is combined with antigen epitopes other than the abnormal decarboxylated site of DCP (abnormal decarboxy prothrombin). Finally, HRP luminescent substrate is added and the luminescence signal is scanned and quantized by a chemiluminescence scanner.

Experimental results show that the method can be applied to detect prothrombin and DCP by luminescence intensity. Compared with ELISA method, both sensitivity and specificity are superior to ELISA method. In terms of time, ELISA detection requires at least 3 hours, and this invention only requires 1.5 hours. In terms of serum requirements, only 10 ul of the original serum or diluted serum is required, while at least 50 ul of the original serum or diluted serum is required by ELISA. Compared with the amount of capture antibody, the amount required is much lower than that of ELISA method. A non-contact inkjet spray point method is applied by the chip of the invention, and each of the detection spots is sprayed with 300-500 pl at a time for 6-10 times. A total of 3 nl point-sample antibodies are sprayed, and the diameter of the detection spot is 0.5-1 mm. Each detection grid has 4 detection points, and 12 nl capture antibodies is required. Point sample uniformity has been guaranteed by precise control of spray volume and a plurality of spray points, effectively reducing the occurrence of hollow phenomenon, greatly improving the detection accuracy, saving the amount of antibody and serum dosage, and reducing the detection cost and cost.

Therefore, the kit and detection method provided by the invention have the characteristics of high sensitivity, time saving and economy, and can greatly reduce the cost and time of blood protein detection.

To sum up, the method of the invention combines the application of abnormal decarboxylation specific antibody, chemiluminescence detection method and protein chip technology to ensure that DCP detection results have high sensitivity, accuracy, high efficiency and low cost by using the kit. The detection method provided by the invention is feasible, reliable, economical, simple and time-saving. The technical scheme of the invention will provide an economical and reliable kit and detection method for large-scale and high-throughput detection of DCP in serum.

### BRIEF DESCRIPTION OF THE DRAWINGS

FIG. 1 is a structure diagram of DCP protein chip, including 1—substrate carrier, 2—detection subregion, 3—detection spot, 4—control spot, 5—physical partition.

FIG. 2 is a flow chart of protein chip by DCP antibody sandwich method.

FIG. 3 is a scanning chart of liver cancer and normal serum samples detected by the DCP antibody point sample chip, including 1-8: liver cancer serum; 9 healthy control serum; 10 blank control.

### DETAILED DESCRIPTION OF THE PREFERRED EMBODIMENTS

The invention is described in details below in combination with the embodiments, but the scope of the invention is not limited. Unless otherwise specified, the operations in the following embodiments are conventional, and the reagents used are commercially available.

### Embodiment 1. Preparation and Validation of Protein Chips 1-4

### Main Equipment

Chemiluminescence scanner, by GE USA.

Main Reagents and their Sources

Murine monoclonal antibody DCP antibody (FUJIREBIO INC, Japan), aldehyde chip (Shanghai BaiO Technology Co., Ltd.), HRP-labeled prothrombin rabbit antibody (Fitzgerald Inc, USA), HRP chemiluminescence substrate liquid A and liquid B mixed in 1:1 and freshly prepared. (Millipore Corporation, USA)

Abnormal prothrombin standard product: FUJIREBIO INC, Japan.

Reagents and instruments used in the experiment: DCP antibody (FUJIREBIO INC, Japan); HRP-labeled rabbit antibody (Fitzgerald Inc., USA); Chemiluminescence scanner (GE, USA)

PBS formula: 8 g sodium chloride (NaCl), 0.2 g potassium chloride (KCl), 1.44 g disodium hydrogen phosphate (Na2HPO4), 0.24 g potassium dihydrogen phosphate (KH2PO4), pH 7.4, constant volume 1 L

PBST formula: PBS, 1 L+tween-20, 1 ml

Substrate carrier 1 is an aldehyde chip (Shanghai BaiO Technology Co., Ltd.), and each chip contains 10 detection squares (detection subregions); one serum sample is detected in each square, and 10 serum samples are detected at a time. The length×width×thickness of each square is 76.4×25.2×1 mm.

### Step 1. Preparing Chips 1-4

In each of the detection subregions 2, DCP antibodies of mice were successively added on the chip, and the DCP antibody point sample concentration was 4 mg/ml.

Each of the detection subregions 2 was provided with a detection spot area and a control spot area. The detection spot area had a detection spot 3 formed by spraying a trace of DCP specific antibody, and the control spot area had a control spot 4 formed by spraying bovine serum albumin at the concentration of 4 mg/ml.

The scheme of pointing the capture antibody was as follows:

Chip 1: in each detection grid, the concentration of murine DCP monoclonal antibody was 4 mg/ml, and 10 ul antibody was applied for each detection spot; 10% bovine serum albumin (BSA) was used as a negative control, 10 ul for each control spot.
Chip 2: the concentration of murine DCP monoclonal antibody was 4 mg/ml, and 5 ul antibody was applied for each detection spot; 10% bovine serum albumin (BSA) was used as a negative control, 5 ul for each control spot.
Chip 3: the concentration of murine DCP monoclonal antibody was 4 mg/ml, and 1 ul antibody was applied for each detection spot; 10% bovine serum albumin (BSA) was used as a negative control, 1 ul for each control spot.
Chip 4: the point sample concentration of murine DCP monoclonal antibody was 4 mg/ml, and 100 nl antibody was applied for each detection spot; 10% bovine serum albumin (BSA) was used as a negative control, 100 nl for each control spot.

Conventional contact pointing method was adopted.

### Step 2. Use of Standard Curve and Standard Product Verification Chip

Preparation of Standard Products:

Standard abnormal prothrombins and serum for testing with a series of gradient concentration were respectively added to normal serum:

Standard serum: 0 mAU/ml, 5 mAU/ml, 10 mAU/ml, 20 mAU/ml, 40 mAU/ml, 80 mAU/ml, 160 mAU/ml, 320 mAU/ml, 640 mAU/ml):

Serum for testing: 0 mAU/ml, 2 mAU/ml, 4 mAU/ml, 8 mAU/ml, 16 mAU/ml, 35 mAU/ml, 50 mAU/ml, 70 mAU/ml, 90 mAU/ml, 120 mAU/ml, 150 mAU/ml, 300 mAU/ml):

### Operation Procedure of Protein Chip:

10 ul sample under test (which can be obtained by diluting 2.5 ul serum 4 times with PBS) was added to each of the chip detection subregion for incubation at 37° C. for 30 minutes, so that the DCP in the serum and DCP antibodies formed antigen-antibody complexes by means of antigen and antibody binding features.

PBST was applied for washing four times to remove nonspecific binding, and the PBS diluted HRP-labeled rabbit primary antibody was added for incubation at 37° C. for 30 minutes. Rabbit antibody was binded with antigen to form DCP antibody-DCP-rabbit HRP-labeled prothrombin antibody complexes.

PBST was applied for washing four times to remove nonspecific binding, HRP luminous substrate was added for incubation at 37° C. for 30 minutes, and detection spot pixel values were obtained by scanning of a chemiluminescence scanner.

The chemiluminescence pixels on the solid phase carrier are positively correlated with the amount of antigen detected in the specimen. The chip capture point-sample antibody (murine primary antibody) and the antibody for detection (rabbit primary antibody) were respectively taken from animals of different species to target different epitopes of prothrombin. As shown in FIG. 2, a flow chart of protein chip by antibody sandwich method was provided.

With the concentration of the standard serum as the horizontal coordinate and pixel values as the vertical coordinate, the standard curve was drawn and the regression equation of the standard curve was generated.

The square value R² of the correlation coefficient of the regression equation of the standard curve obtained by chips 1-4 was between 0.55 and 0.6, and the linear correlation between the concentration of the standard product and the pixel value was poor.

Another batch of standard serum for testing with different concentrations was used to verify the standard curve, and it was found that the results obtained were significantly different from the concentration of standard serum for testing itself, and the test values of the standard serum for testing with a concentration lower than 80 mAU/ml were repeatedly lower than 30 mAU/ml, which was lower than the diagnostic threshold value of 40 mAU/ml, showing a false negative.

Step 3. These chips was validated with clinical serum having a given DCP content, with the serum samples as follows:

35 samples of liver cancer serum are collected from the specimen bank of You'an Hospital, Capital Medical University. It is known that the abnormal decarboxy prothrombin's concentration of these samples is higher than the diagnostic threshold value 40 mAU/ml (1 mAU/ml=1 ng/ml).

28 normal healthy human serum samples; the concentration of abnormal decarboxy prothrombin is lower than the diagnostic threshold value.

7 blank controls (blank control is 1×PBS), and the test results are statistically shown in table 1 below:

TABLE 1
Detection results of chips 1-4

| | Chip 1 | | Chip 2 | | Chip 3 | | Chip 4 | |
|---|---|---|---|---|---|---|---|---|
| | DCP ≥ 40 | DCP < 40 | DCP ≥ 40 | DCP < 40 | DCP ≥ 40 | DCP < 40 | DCP ≥ 40 | DCP < 40 |
| | mAU/ml | mAU/ml | mAU/ml | mAU/ml | mAU/ml | mAU/ml | mAU/ml | mAU/ml |
| Liver | 20 | 15 | 21 | 14 | 24 | 11 | 24 | 11 |
| cancer | | | | | | | | |
| serum | | | | | | | | |
| (35) | | | | | | | | |
| Healthy | 0 | 28 | 0 | 28 | 0 | 28 | 0 | 28 |
| serum | | | | | | | | |
| (28) | | | | | | | | |
| Blank | 0 | 7 | 0 | 7 | 0 | 7 | 0 | 7 |
| control | | | | | | | | |
| (7) | | | | | | | | |

As can be seen from the results in table 1, DCP is not detected in 7 blank control samples. No DCP is detected in 28 healthy serum samples. Only 20-24 of the 35 samples of HCC serum are detected with DCP higher than the critical value, that is, these chips have detection sensitivity of (20-24)/35=57.1%-68.6%, and the proportion of missed detection is very high.

### Embodiment 2. Preparation and Validation of Chips 5-7

### Step 1: Preparing the Chips

Chip 5: the point sample concentration of murine DCP monoclonal antibody was 4 mg/ml, and 10 nl antibody was applied for each detection spot; 10% bovine serum albumin (BSA) was used as a negative control, 10 nl for each control spot;

Control chip 6: the concentration of murine DCP monoclonal antibody was 4 mg/ml, and 5 nl antibody was applied for each detection spot; 10% bovine serum albumin (BSA) was used as a negative control, 5 nl for each control spot.

Control chip 7: the concentration of murine DCP monoclonal antibody was 4 mg/ml, and 2 nl antibody was applied for each detection spot; 10% bovine serum albumin (BSA) was used as a negative control, 2n1 for each control spot;

The control chips 5-7 adopted a non-contact point sampler with picoliter precision, i.e. Nano-Plotter NP2.1 of GESIM Company, Germany, with a piezoelectric point sampler needle, and the temperature of the cabin inside the point sampler controlled to be within 4-8 degrees Celsius.

### Step 2: Procedure and Experimental Reagent Same as Embodiment 1.

The square value R² of the correlation coefficient of the regression equation of the standard curve obtained by chips 5-7 was gradually increased to 6.5-7.5, indicating that the linear relationship between the concentration of the standard product and the pixel value was significantly improved.

Another batch of standard serum for testing with different concentrations was used to verify the standard curve, and it was found that the difference between the obtained results and the test standard serum concentration was reduced, and the minimum detection limit was up to 4 mAU/ml.

### Step 3. Chip Validation by Given Clinical Serum

Serum is the same as step 3 of Embodiment 1, and the test results are statistically shown in table 2.

TABLE 2
Detection results of chip 5-7

| | Chip 8 | | Chip 9 | |
|---|---|---|---|---|
| | DCP ≥ 40 | DCP < 40 | DCP ≥ 40 | DCP < 40 |
| | mAU/ml | mAU/ml | mAU/ml | mAU/ml |
| Liver cancer | 27 | 8 | 30 | 5 |
| serum (35) | | | | |
| Healthy serum | 0 | 28 | 0 | 28 |
| (28) | | | | |
| Blank control | 0 | 7 | 0 | 7 |
| (7) | | | | |

As can be seen from the results in table 2, DCP is not detected in 7 blank control samples. No DCP is detected in 28 healthy serum samples. DCP is detected in 27-30 of the 35 samples of liver cancer serum higher than the critical value, that is, these chips have detection sensitivity of (27-30)/35=77.1%-85.7%, and the detection rate is significantly improved, but there is still a considerable proportion of missed detection. Combined with the verification of the test standard serum in step 2, it can be preliminarily determined that the detection is missed due to insufficient detection limit.

In parallel embodiments, the point volume is further reduced, but no improvement in the linear relationship between the concentration of the standard substance and the pixel value is further observed in the standard curve obtained from preparation. The detection of test standard serum and clinical serum is not significantly improved compared with chips 5-7.

### Embodiment 3. Fabrication and Verification of Chips 8-10

With multi-dimensional attempts such as antibody purification and reagent formulation to detection temperature and time, the inventor has not observed further improvement in the linear relationship between standard substance concentration and pixel value, and the detection rate is difficult to break through 90%. In the accidental experiment, the inventor tried to divide the antibody into several parts and repeat pointing by several times on one spot, the resulted chip unexpectedly jumped to 0.9-0.95 in the correlation coefficient R² of the standard curve and 92-95% in the detection rate relative to the other conditions unchanged in Embodiment 2, as follows:

### Step 1. Fabrication of Chips 8 and 9

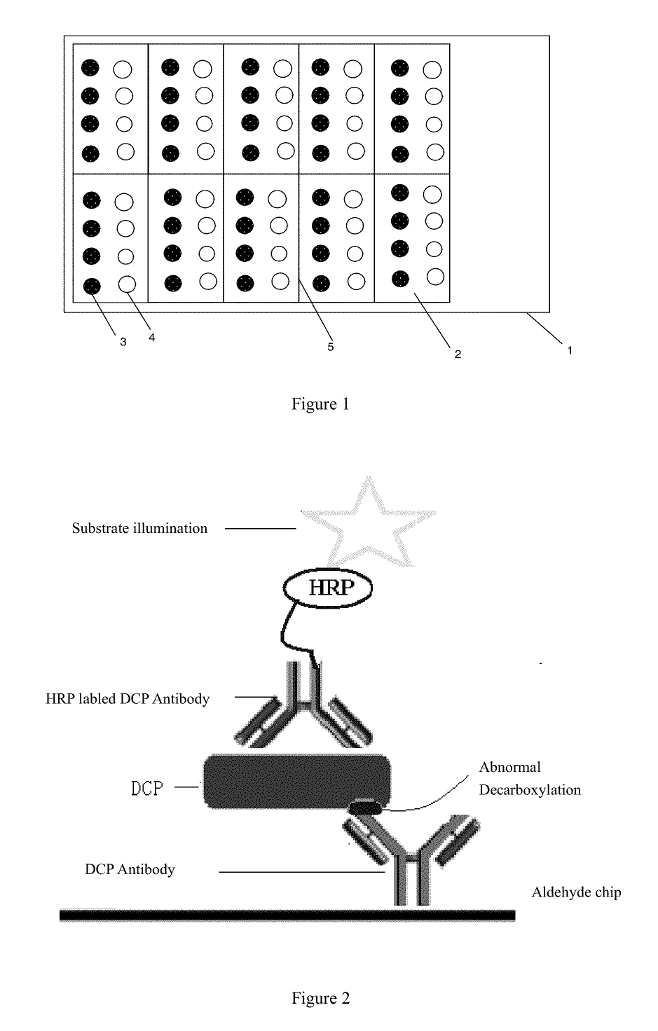

Chip 8: the spot concentration of murine DCP monoclonal antibody was 4 mg/ml, and 3 nl antibody was applied for each detection spot; 10% bovine serum albumin (BSA) was used as a negative control, 5 nl for each control spot. The non-contact point sampler with picoliter precision, i.e. Nano-Plotter NP2.1 of GESIM Company, Germany was adopted, with a piezoelectric point sampler needle, and the temperature of the point sampler inner chamber controlled to 4-8 degrees Celsius. Each point was sprayed with 300 pl-500 pl at a time for 3-5 times, with a total of 3 nl antibodies sprayed, and the diameter of the detection spot was 0.5-1 mm. The chip's structure was as shown in FIG. 1.

Chip 9: the point sample concentration of murine DCP monoclonal antibody was 4 mg/ml, and 5 nl antibody was detected for each spot; 10% bovine serum albumin (BSA) was used as a negative control, 5 nl for each control spot. The non-contact point sampler with picoliter precision, i.e. Nano-Plotter NP2.1 of GESIM Company, Germany was adopted, with a piezoelectric point sampler needle, and the temperature of the point sampler inner chamber controlled to 4-8 degrees Celsius. Each point was sprayed with 300 pl-500 pl at a time for 6-10 times, with a total of 3 nl point-sample antibodies sprayed, and the diameter of the detection spot was 0.5-1 mm.

### Step 2. Procedure and Experimental Reagent Same as Embodiment 1

The square value R² of the correlation coefficient of the regression equation of the standard curve obtained by chip 8 or 9 jumped to 9.2-9.5, and the linear relationship between the concentration of the standard product and the pixel value was significantly improved.

Another batch of test standard serum with different concentrations was used to verify the standard curve, and it was found that the result obtained was only slightly different from the concentration of the test standard serum itself, which was within the allowable range of error, wherein the test result of the test standard serum with a concentration of 60 mAU/ml was lower than the defined value, showing a false negative.

### Step 3. Chip Validation by Given Clinical Serum

Serum is the same as step 3 of Embodiment 1, and the test results are statistically shown in table 3.

TABLE 3
Detection results of chips 8 and 9

| | Chip 8 | | Chip 9 | |
|---|---|---|---|---|
| | DCP ≥ 40 | DCP < 40 | DCP ≥ 40 | DCP < 40 |
| | mAU/ml | mAU/ml | mAU/ml | mAU/ml |
| Liver cancer | 33 | 2 | 34 | 1 |
| serum (35) | | | | |
| Healthy serum | 0 | 28 | 0 | 28 |
| (28) | | | | |
| Blank control | 0 | 7 | 0 | 7 |
| (7) | | | | |

As can be seen from the results in table 1, DCP is not detected in 7 blank control samples. No DCP is detected in 28 healthy serum samples. DCP is detected in 33 of the 35 samples of HCC serum higher than the critical value, that is, the detection sensitivity of these chips (33-34)/35=94.3%-97.1%, and the detection rate is almost 100%.

The optimal chip scheme is as follows:

In each detection grid (detection subregion), DCP antibody of rats was successively added on the chip. The DCP antibody sample concentration was 4 mg/ml, and four detection spots were added in a row. 10% bovine serum albumin (BSA) was used as a negative control, and also added four times to form control spots;

The non-contact point sampler with picoliter precision, i.e. Nano-Plotter NP2.1 of GESIM Company, Germany was adopted, with a piezoelectric point-sample needle, and the temperature of the inner chamber of the point-sample instrument controlled to 4-8 degrees Celsius. Each point was sprayed with 300 pl-500 pl at a time for 6-10 times, with a total of 3-5 nl point-sample antibody sprayed. Each detection area was provided with 4 detection points, requiring 12 nl capture antibody, and the diameter of detection spot was 0.5-1 mm.

### Experiment Application Effect of Optimal Chip Scheme

The correlation coefficient R² of the standard curve regression equation is 0.98.

### Serum Samples:

8 samples of liver cancer serum are collected from the specimen bank of Beijing You'an Hospital Affiliated to Capital Medical University (the known abnormal decarboxy prothrombin concentration is higher than the diagnostic threshold value 40 mAU/ml (1 mAU/ml=1 ng/ml).

1 normal healthy human serum;

1 blank control (blank control is 1×PBS).

### Detection Results of the Chip:

No abnormal prothrombin is detected in the blank control samples and healthy control samples: indicating that the chip used in this experiment is effective. There is no abnormal prothrombin in normal people and no prothrombin is detected in healthy serum. This means that the false positive detected by the chip and method provided by the invention is 0, and the detection result can accurately distinguish the serum of liver cancer from the normal serum.

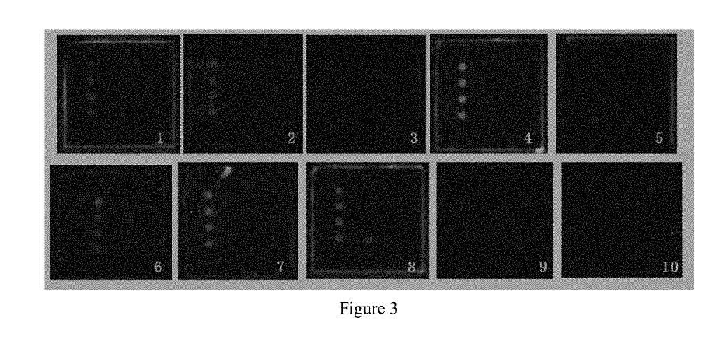

The results of spot scanning and calculation of 8 samples of HCC serum show positive, indicating that abnormal decarboxy prothrombin is found in 8 samples of HCC serum, especially in patients with advanced HCC; the detection spots in the 8 positive detection subregions show different degrees of brightness, indicating that the 8 liver cancer serum contain different concentrations of abnormal decarboxy prothrombin, as shown in FIG. 3, wherein 3 and 5 show weak positive, and the detection spot brightness is weak, while 1, 2, 4, 6, 7, 8 show strong positive. The detection results indicate that the protein chip of the invention can accurately detect the serum of liver cancer and semi-quantitatively detect the concentration of abnormal decarboxy prothrombin in the serum. The above data shows that the chip and method of the invention are accurate and reliable.

In addition, the sensitivity and specificity of serum tests in dozens of outpatients with liver cancer are more than 90%.

### The Kit of the Invention:

Including Chips shown in Embodiment 3, wherein a protrusion is provided between the preferable detection subregions 2 in some chips as a physical partition 5; and

HRP-labeled prothrombin polyclonal antibody, and HRP chemiluminescent substrate liquid; the
HRP-labeled prothrombin polyclonal antibody is a rabbit antibody, and the DCP-specific antibody fixed on the detection spot is originated from a different species.

1. A high-throughput protein chip for detecting abnormal decarboxy prothrombin in serum, characterized in that:
a plurality of detection subregions are provided on a substrate carrier of the protein chip, and each of the detection subregions is configured to detect a serum sample; each of the detected subregions is provided with a detection spot area and a control spot area, the detection spot area has a detection spot formed by spraying a trace of DCP specific antibody, and the control spot area has a control spot formed by spraying bovine serum albumin; substances on all the detection spots have the same concentration in the same detection spot area; and
the total amount of DCP specific antibody for forming each of the detection spots is 3-5 nl, and the concentration thereof is 3-5 mg/ml, each of the detection spots is formed by a non-contact point sampler in 6-10 times and spraying 300-500 pl each time, and the diameter of the detection spot is 0.5-1 mm.

2. The protein chip of claim 1, characterized in that each of the detection spot areas includes 4-8 mutually separated detection spots arranged in a row, and the control spot area includes 4-8 mutually and independently separated control spots arranged in a row; the detection spots and the control spots are arranged in two parallel columns.

3. The protein chip of claim 2, characterized in that the length, width and thickness of the chip are 76.4 mm, 25.2 mm and 1 mm; and 10 detection subregions are provided on the substrate carrier.

4. The protein chip of claim 2, characterized in that a protrusion is provided between the detection subregions as a physical partition.

5. The protein chip of claim 1, characterized in that the specific antibody of the DCP is a murine anti-human DCP.

6. A method of preparing the protein chip for detecting abnormal decarboxy prothrombin of any of claims 1 to 5, characterized in that:
the total amount of DCP-specific antibody for forming each of the detection spots is 3 nl, and the concentration thereof is 4 mg/ml; a detection spot is formed by spraying the DCP-specific antibody in 6-10 times and 300-500 pl each time.

7. The preparation method of claim 6, characterized in that the temperature of the DCP-specific antibody is 4-8° C. during the spraying process.

8. The preparation method of claim 6 or 7, characterized in that spraying the antibody is performed by the non-contact point sampler.

9. (canceled)

10. A method for detecting abnormal decarboxy prothrombin in serum, characterized in that the use of protein chip of claims 1-6 comprises the steps of:
diluting the serum samples to be tested and dropping onto the detection subregion of the protein chip; after incubation, washing the detection subregion with PBST to remove nonspecific binding substances.
adding HRP-labeled prothrombin antibody diluted with PBS; after incubation, washing with PBST to remove the nonspecific binding substances; and
adding HRP substrate luminescent solution, scanning the protein chip by a chemiluminescence scanner to obtain DCP luminescence pixel values in serum samples to be measured after dilution respectively;
wherein the incubation refers to incubating at 37° C. for 30 minutes.

11. The preparation method of claim 7, characterized in that spraying the antibody is performed by the non-contact point sampler.

12. A method for detecting abnormal decarboxy prothrombin in serum, characterized in that the use of protein chip of claim 2 comprises the steps of:
diluting the serum samples to be tested and dropping onto the detection subregion of the protein chip; after incubation, washing the detection subregion with PBST to remove nonspecific binding substances.
adding HRP-labeled prothrombin antibody diluted with PBS; after incubation, washing with PBST to remove the nonspecific binding substances; and
adding HRP substrate luminescent solution, scanning the protein chip by a chemiluminescence scanner to obtain DCP luminescence pixel values in serum samples to be measured after dilution respectively;
wherein the incubation refers to incubating at 37° C. for 30 minutes.

13. A method for detecting abnormal decarboxy prothrombin in serum, characterized in that the use of protein chip of claim 3 comprises the steps of:
diluting the serum samples to be tested and dropping onto the detection subregion of the protein chip; after incubation, washing the detection subregion with PBST to remove nonspecific binding substances.
adding HRP-labeled prothrombin antibody diluted with PBS; after incubation, washing with PBST to remove the nonspecific binding substances; and
adding HRP substrate luminescent solution, scanning the protein chip by a chemiluminescence scanner to obtain DCP luminescence pixel values in serum samples to be measured after dilution respectively;
wherein the incubation refers to incubating at 37° C. for 30 minutes.

14. A method for detecting abnormal decarboxy prothrombin in serum, characterized in that the use of protein chip of claim 4 comprises the steps of:
diluting the serum samples to be tested and dropping onto the detection subregion of the protein chip; after incubation, washing the detection subregion with PBST to remove nonspecific binding substances.
adding HRP-labeled prothrombin antibody diluted with PBS; after incubation, washing with PBST to remove the nonspecific binding substances; and
adding HRP substrate luminescent solution, scanning the protein chip by a chemiluminescence scanner to obtain DCP luminescence pixel values in serum samples to be measured after dilution respectively;
wherein the incubation refers to incubating at 37° C. for 30 minutes.

15. A method for detecting abnormal decarboxy prothrombin in serum, characterized in that the use of protein chip of claim 5 comprises the steps of:
diluting the serum samples to be tested and dropping onto the detection subregion of the protein chip; after incubation, washing the detection subregion with PBST to remove nonspecific binding substances.
adding HRP-labeled prothrombin antibody diluted with PBS; after incubation, washing with PBST to remove the nonspecific binding substances; and
adding HRP substrate luminescent solution, scanning the protein chip by a chemiluminescence scanner to obtain DCP luminescence pixel values in serum samples to be measured after dilution respectively;
wherein the incubation refers to incubating at 37° C. for 30 minutes.

16. A method for detecting abnormal decarboxy prothrombin in serum, characterized in that the use of protein chip of claim 6 comprises the steps of:
diluting the serum samples to be tested and dropping onto the detection subregion of the protein chip; after incubation, washing the detection subregion with PBST to remove nonspecific binding substances.
adding HRP-labeled prothrombin antibody diluted with PBS; after incubation, washing with PBST to remove the nonspecific binding substances; and
adding HRP substrate luminescent solution, scanning the protein chip by a chemiluminescence scanner to obtain DCP luminescence pixel values in serum samples to be measured after dilution respectively;
wherein the incubation refers to incubating at 37° C. for 30 minutes.
